(19) United States
(12) Patent Application Publication
(10) Pub. No.: US 2023/0108635 A1
(43) Pub. Date: Apr. 6, 2023

(54) BIOLOGICAL ELECTRODE

(71) Applicant: TATSUTA ELECTRIC WIRE & CABLE CO., LTD., Osaka (JP)

(72) Inventors: Takashi MORITA, Osaka (JP); Tatsuya SAITOU, Osaka (JP)

(21) Appl. No.: 17/908,469

(22) PCT Filed: Nov. 10, 2020
(86) PCT No.: PCT/JP2020/041836

§ 371 Date: Aug. 31, 2022

(30) Foreign Application Priority Data
Mar. 13, 2020 (JP) 2020-044436

(51) Int. Cl. A61B 5/25

(52) U.S. Cl. CPC A61B 5/25; A61B 2562/0209; A61B 2562/125

(57) ABSTRACT

Provided is a biological electrode including: a polarizable electrode layer; and a non-polarizable electrode layer laminated on the polarizable electrode layer, in which the non-polarizable electrode layer includes a resin, silver, and silver chloride supported by silica, a content of the silver is 150 to 300 mass parts based on 100 mass parts of the resin, a ratio between the silver and the silver chloride is 97:3 to 95:5, and the non-polarizable electrode layer has a thickness of 3 to 5 μm.

### CROSS-REFERENCE TO RELATED APPLICATION

This application claims priority to Japanese Patent Application No. 2020-044436, the disclosure of which is incorporated herein by reference in its entirety.

### FIELD

The present invention relates a biological electrode.

### BACKGROUND

Conventionally used has been a biological electrode including: a polarizable electrode layer; and a non-polarizable electrode layer including silver and silver chloride.

Such a biological electrode is used by being attached to a patient in, for example, electrocardiography. In so doing, there are some cases where an X-ray image is taken with the biological electrode attached to the patient. It is therefore preferable that the biological electrode have a reduced amount of silver for an improved X-ray transmittance so as not to be captured in an X-ray image. In addition, it is preferable that the biological electrode have a reduced amount of silver in terms also of being produced at low cost.

Known as such a biological electrode is, for example, a biological electrode in Patent Literature 1 that includes a non-polarizable electrode layer formed of a thin silver/silver chloride film into a thickness of several micrometers by a method such as printing or deposition on a polarizable electrode layer.

Also known is a biological electrode that includes a non-polarizable electrode layer formed of a resin composition in which silver/silver chloride is dispersed in a resin, in order to further reduce the amount of silver/silver chloride. As such a biological electrode, for example, Patent Literature 2 discloses a biological electrode including a non-polarizable electrode layer (highly-porous uppermost conductive layer) formed of a conductive coating including a resin, silver/silver chloride particles, and carbon particles. The biological electrode configured as above includes the highly porous non-polarizable electrode layer to apparently allow a low content of silver/silver chloride particles to exert required electrical properties.

### CITATION LIST

### Patent Literature

Patent Literature 1: JP H5-095922 A

Patent Literature 2: JP H10-248820 A

### SUMMARY

### Technical Problem

However, the conventional biological electrodes have a problem that the silver content is not sufficiently reduced, resulting in insufficient X-ray transmittance in particular. Another problem is that an attempt to reduce the silver content using the conventional technique decreases the electrical performance of the biological electrode. For example, a biological electrode produced using carbon particles has a problem of reduced non-polarizability resulting in insufficient electrical performance, and for example, there are some cases where such a biological electrode fails to sufficiently satisfy the American National Standard (ANSI/AAMI EC12:2000/(R) 2010).

In view of the above problems, it is an object of the present invention to provide a biological electrode having a reduced silver content but still excellent in electrical performance.

### Solution to Problem

A biological electrode according to the present invention includes: a polarizable electrode layer; and a non-polarizable electrode layer laminated on the polarizable electrode layer, in which the non-polarizable electrode layer includes a resin, silver, and silver chloride supported by silica, a content of the silver is 150 to 300 mass parts based on 100 mass parts of the resin, a ratio between the silver and the silver chloride is 97:3 to 95:5, and the non-polarizable electrode layer has a thickness of 3 to 5 μm.

In the biological electrode according to the present invention, it is preferable that a content of the silica be 20 to 50 mass parts based on 100 mass parts of the resin.

In the biological electrode according to the present invention, it is preferable that the polarizable electrode layer have a thickness of 5 to 10 μm.

### BRIEF DESCRIPTION OF DRAWINGS

FIG. 1 is a schematic view showing a sectional structure of a biological electrode according to one embodiment.

FIG. 2 shows graphs of the results shown in Table 1 in Examples.

FIG. 3 shows graphs of the results shown in Table 3 in Examples.

### DESCRIPTION OF EMBODIMENTS

A description will be hereinafter given on a biological electrode according to one embodiment of the present invention with reference to the drawings.

A biological electrode 1 of this embodiment is used to acquire biological information such as an electrocardiogram, an electroencephalogram, and an electromyogram. Thus, it is preferable that the biological electrode 1 sufficiently satisfy the American National Standard (ANSI/AAMI EC12:2000/(R) 2010). For example, the impedance of the biological electrode 1 (hereinafter referred to also as ACZ) is preferably 2000Ω or less, more preferably 1000Ω or less, further preferably 750Ω or less. The polarization of the biological electrode 1 after defibrillation load (which serves as an index of non-polarizability and is hereinafter referred to also as SDR/DCO) is preferably 100 mV or less, more preferably 20 mV or less. The biological electrode 1 preferably has storage stability, and can, for example, preferably maintain the above electrical performance over 5 weeks or more or 10 weeks or more under the temperature condition of 57° C. Further, the biological electrode 1 is configured to be suitably used when an X-ray image is taken in parallel with acquiring the above biological information.

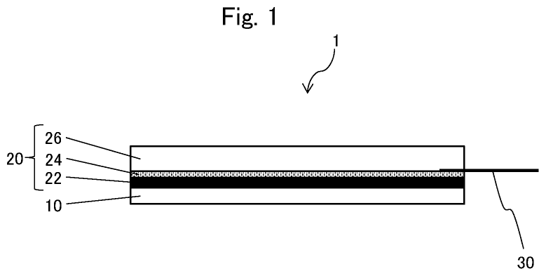

As shown in FIG. 1, the biological electrode 1 of this embodiment includes a substrate 10 having a sheet shape, and an electrode layer 20 laminated on one surface of the substrate 10 and configured to detect electrical signals from a living body. The biological electrode 1 is used with a lead wire 30 connected to the electrode layer 20.

The substrate 10 is generally formed of an electrically insulating film made of polyethylene terephthalate (PET). An electrically insulating film herein refers to a film having a volume resistivity of 1×10¹³Ω·cm or more. The thickness of the substrate 10 is generally set to 5 to 100 μm.

As the lead wire 30, a conventionally known lead wire can be used. The lead wire 30 can be formed of, for example, a metal conductor and a cover material covering the metal conductor. Also, it is preferable that the lead wire 30 have a conductor formed of conductive carbon fibers bundled together in substitution for the metal conductor, so as to be hardly captured in X-ray images. The conductor of the lead wire 30 has one end electrically connected to the electrode layer 20.

The electrode layer 20 includes a polarizable electrode layer 22 formed on one surface of the substrate 10, a non-polarizable electrode layer 24 formed on one surface of the polarizable electrode layer 22, and a conductive gel layer 26 formed on one surface of the non-polarizable electrode layer 24. In this embodiment, the lead wire 30 is disposed between the non-polarizable electrode layer 24 and the conductive gel layer 26.

In this embodiment, the polarizable electrode layer 22 is formed of a carbon paste including graphite and/or carbon powder dispersed a resin.

The thickness of the polarizable electrode layer 22 is generally 2 to 100 μm. In terms of sufficiently reducing the ACZ of the biological electrode 1, the thickness of the polarizable electrode layer 22 is preferably 5 μm or more. Further, in terms of being capable of producing the biological electrode 1 at low cost, the thickness of the polarizable electrode layer 22 is preferably 10 μm or less.

The non-polarizable electrode layer 24 includes a resin, silver, and silver chloride supported by silica. That is, the non-polarizable electrode layer 24 is formed by, for example, printing, transferring, or applying a resin composition in which silver and silica supporting silver chloride are dispersed in a resin to one surface of the polarizable electrode layer 22 to thereby cover the one surface. Silver chloride supported by silica is hereinafter sometimes referred to as silver chloride supporting silica.

The thickness of the non-polarizable electrode layer 24 is preferably more than 2 μm and less than 8 more preferably 3 to 5 This allows the biological electrode 1 to have excellent X-ray transmittance while maintaining its electrical performance.

The thickness of each of the polarizable electrode layer 22 and the non-polarizable electrode layer 24 is calculated as an average thickness by cross-sectional observation with a scanning electron microscope (SEM). Examples of a specific measurement method include a method in which the conductive gel layer 26 is removed from the biological electrode 1; a piece is cut out of the electrode layer 20 by, for example, microtome; the cross section of the cut piece is observed by an SEM at a magnification of 10000 times or less; the thickness of each of the polarizable electrode layer 22 and the non-polarizable electrode layer 24 is measured at a plurality of given positions (for example 9 positions); and an arithmetic average value of the measured thicknesses is used as the average thickness.

Examples of the resin include thermoplastic resins such as a polystyrene resin, a polyester resin, a polyurethane resin, an acrylic resin, an alkyd resin, a phenoxy resin, a butyral resin, or a polyvinyl alcohol resin.

As the silver, silver powder can be used. The shape of the silver powder is not particularly limited, but is preferably spherical or scaly. The silver powder is preferably subjected to surface treatment with a fatty acid such as stearic acid, thereby achieving an increased dispersibility in the resin.

The particle size of the silver is generally 4 μm or less, preferably 3 μm or less, more preferably 2 μm or less, so that the non-polarizable electrode layer 24 can be set to have a small thickness. The particle size of the silver means a median size (D50) thereof measured by a laser diffraction scattering type particle size distribution measuring device.

The content of the silver is preferably more than 140 mass parts, more preferably 150 mass parts or more, further preferably 180 mass parts or more, based on 100 mass parts of the resin. This sufficiently reduces the ACZ of the biological electrode 1. The content of the silver is preferably 300 mass parts or less, more preferably 250 mass parts or less, further preferably 200 mass parts or less. This allows the biological electrode 1 to have excellent X-ray transmittance.

As the silver chloride supporting silica, a silver chloride supporting silica prepared according to, for example, the method described in WO 2018/003702 A is used. Since the silver chloride is supported by the silica, the silver chloride can be prevented from aggregating and can be made present uniformly in the non-polarizable electrode layer 24.

It is preferable that the particle size of the silver chloride supporting silica be preferably 4 μm or less, more preferably 3 μm or less, so that the non-polarizable electrode layer 24 can be set to have a small thickness. The particle size of the silver chloride supporting silica means a median size (D50) thereof measured by a laser diffraction scattering type particle size distribution measuring device.

A conventionally known silica can be used as the silica. The silica can be a wet-method silica such as a precipitation silica or a gelled silica, and can be a dry silica, among which a gelled silica is preferable.

The specific surface area of the silica is preferably 20 m²/g or more and 1000 m²/g or less, more preferably 400 m²/g or more and 700m²/g or less. The specific surface area means the value measured by the BET method based on inert gas adsorption.

The pore volume of the silica is preferably 0.2 mL/g or more and 2.0 mL/g or less, more preferably 0.3 mL/g or more and 0.6 mL/g or less. The pore volume means the value measured by a mercury porosimeter.

The oil absorption of the silica is preferably 50 mL/100 g or more and 500 mL/100 g or less, more preferably 70 mL/100 g or more and 170 mL/100 g or less. The oil absorption means the value determined by the test method according to JIS K 5101-13-1.

The average pore diameter of the silica is preferably 2 nm or more and 100 nm or less, more preferably 2 nm or more and 7 nm or less. The average pore diameter means the value measured by a mercury porosimeter.

The particle size of the silica is generally 4 μm or less, preferably 3 μm or less, so that the non-polarizable electrode layer 24 can be set to have a small thickness. The particle size of the silica means a median size (D50) thereof measured by a laser diffraction scattering type particle size distribution measuring device.

The content of the silica is generally 20 to 50 mass parts, preferably 24 to 40 mass parts, based on 100 mass parts of the resin. As will be described later, in this embodiment, a dispersion in which the resin, the silver, and the silver chloride supporting silica are dispersed in an organic solvent is prepared when the non-polarizable electrode layer 24 is formed. In order to uniformly disperse the silver in the non-polarizable electrode layer 24, it is preferable that the dispersion have such a viscosity as to suppress sedimentation of the silver contained therein. In order to increase the viscosity of the dispersion, the content of the silica is preferably 20 mass parts or more, more preferably 24 mass parts or more, based on 100 mass parts of the resin.

The amount of the silver chloride to be used is preferably minimized. This configuration reduces contamination of the equipment for preparing the resin composition and the equipment for coating or other operations. The configuration also reduces use of halogen substances in producing the biological electrode 1, and is therefore environmentally preferable. In terms of maintaining the electrical performance of the biological electrode 1 while reducing the amount of the silver chloride for use, it is preferable that the ratio between the content of the silver and the content of the silver chloride be 97:3 to 95:5.

In consideration of the content of the silica and the content of the silver chloride, the content of the silver chloride relative to the total amount of the silver chloride supporting silica is generally 5 to 45%, preferably 10 to 30%, more preferably 15 to 25%.

The conductive gel layer 26 is a layer that brings the biological electrode 1 into contact with the surface of the skin. The conductive gel layer 26 includes, as an electrolyte, an alkali salt halide such as sodium chloride. A conventionally known gel layer can be used for the conductive gel layer 26.

Next, a description will be given on a method for producing the biological electrode 1, particularly a method for forming the polarizable electrode layer 22 and the non-polarizable electrode layer 24.

The method for producing the biological electrode 1 includes a first layer forming step S1 of forming the polarizable electrode layer 22 on one surface of the substrate 10, and a second layer forming step S2 of forming the non-polarizable electrode layer 24 on one surface of the polarizable electrode layer 22.

The first layer forming step S1 is a step of coating the one surface of the substrate 10 with a carbon paste prepared by dispersing a resin and a graphite and/or carbon powder in an organic solvent, followed by removing the organic solvent by drying to form the polarizable electrode layer 22.

The second layer forming step S2 includes a step of preparing a dispersion in which silver and silver chloride supporting silica are dispersed in a resin, and a step of covering the surface of the polarizable electrode layer 22 with the dispersion to form the non-polarizable electrode layer 24.

In the step of preparing the dispersion, the dispersion is prepared by dissolving the resin in an organic solvent, dispersing the silver and the silver chloride supporting silica therein, and stirring the intermediate at ordinary temperature.

Preferable examples of the organic solvent include a ketone-based solvent such as methyl ethyl ketone (MEK) and cyclohexanone, an ester-based solvent such as ethyl acetate, a cyclic alkane-based solvent such as cyclohexane and methylcyclohexane, and an aromatic solvent having one benzene ring such as benzene, xylene (including isomeric o-xylene, m-xylene, and p-xylene), and toluene. In addition, a linear or branched alkane-based solvent having 5 to 8 carbon atoms is also preferably used. Examples of such a solvent include: n-pentane, 2-methylbutane, and 2,2-dimethylpropane, each of which has 5 carbon atoms; n-hexane, 3-methylpentane, 2-methylpentane, 2,3-dimethylbutane, and 2,2-dimethylbutane, each of which has 6 carbon atoms; n-heptane, 2-methylhexane, 3-methylhexane, 3-ethylpentane, 2,2-dimethylpentane, 2,3-dimethylpentane, 2,4-dimethylpentane, 3,3-dimethylpentane, and 2,2,3-trimethylbutane, each of which has 7 carbon atoms; and n-octane, 2-methylheptane, 3-methylheptane, 4-methylheptane, 2,2-dimethylhexane, 2,3-dimethylhexane, 2,4-dimethylhexane, 2,5-dimethylhexane, 3,3-dimethylhexane, 3,4-dimethylhexane, 3-ethylhexane, 2,2,3-trimethylpentane, 2,2,4-trimethylpentane, 2,3,3-trimethylpentane, 2,3,4-trimethylpentane, 2-methyl-3-ethylpentane, 3-methyl-3-ethylpentane, and 2,2,3,3-tetramethylbutane, each of which has 8 carbon atoms. When the viscosity of the dispersion excessively decreases, the silver tends to settle in the dispersion, making it difficult to uniformly disperse the silver in the non-polarizable electrode layer 24. Thus, the content of the organic solvent relative to the total amount of the dispersion is preferably 60 to 70 mass %.

In the step of forming the non-polarizable electrode layer 24, the surface of the polarizable electrode layer 22 is covered with the dispersion. Examples of the method for covering include printing, transferring, or applying. Since the dispersion of this embodiment has an increased viscosity by containing silica, sedimentation of the silver is suppressed at the time of, for example, printing. This makes it possible to uniformly disperse the silver in the non-polarizable electrode layer 24.

Next, the organic solvent is removed by drying to form the non-polarizable electrode layer 24. The dispersion with an increased viscosity as described above suppresses sedimentation of the silver even at the time of the drying.

As described above, a biological electrode 1 according to this embodiment includes: a polarizable electrode layer 22; and a non-polarizable electrode layer 24 laminated on the polarizable electrode layer 22, in which the non-polarizable electrode layer 24 includes a resin, silver, and silver chloride supported by silica, a content of the silver is 150 to 300 mass parts based on 100 mass parts of the resin, a ratio between the silver and the silver chloride is 97:3 to 95:5, and the non-polarizable electrode layer 24 has a thickness of 3 to 5

According to such a configuration that the non-polarizable electrode layer 24 includes silver and silver chloride in the aforementioned ranges and has a thickness of 3 to 5 the biological electrode 1 having a reduced silver content but still excellent in electrical performance can be achieved.

In the biological electrode 1 according to this embodiment, it is preferable that a content of the silica be 20 to 50 mass parts based on 100 mass parts of the resin.

According to such a configuration that the content of the silica is 20 to 50 mass parts, the dispersion that includes the resin, the silver, and the silica and that is, for example, coated on the polarizable electrode layer 22 to form the non-polarizable electrode layer 24 has a high viscosity. This configuration allows the silver to hardly settle in the dispersion and has an increased dispersibility in the non-polarizable electrode layer 24. Thus, the biological electrode 1 more excellent in electrical performance can be achieved.

In the biological electrode 1 according to this embodiment, it is preferable that the polarizable electrode layer 22 have a thickness of 5 to 10

According to such a configuration that the non-polarizable electrode layer 22 has a thickness of 5 to 10 the biological electrode 1 further excellent in electrical performance can be achieved.

One embodiment has been described as an exemplification, but the biological electrode according to the present invention is not limited to the configuration of the aforementioned embodiment. Further, the biological electrode according to the present invention is not limited by the aforementioned operational effects, either. Various modifications can be made to the biological electrode according to the present invention without departing from the gist of the present invention.

### EXAMPLES

Hereinafter, the present invention will be further described by way of Examples.

### Example

A conductive carbon paste (Varniphite UCC-2 manufactured by Nippon Graphite Industries, Co., Ltd.) was applied across the entire one surface of a PET film (having a thickness of 38 μm) as a substrate, followed by being allowed to dry at 120° C. to thereby form a polarizable electrode layer having a thickness of 5 μm on the one surface of the substrate.

Next, a non-polarizable electrode layer was formed to have the mixing ratio shown in Table 1. The specific steps are provided below.

40 g of silver nitrate was dissolved in 50 mL of ion-exchanged water to prepare an aqueous silver nitrate solution. 20 g of silica (trade name: SYLYSIA 710 manufactured by FUJI SILYSIA CHEMICAL LTD., particle size: 2.8 μm, specific surface area: 700 m²/g, pore volume: 0.44 mL/g, oil absorption: 100 mL/100 g, average pore diameter: 2.5nm) was added to the aqueous silver nitrate solution, which was then stirred for four hours. The solid content was filtered and washed with ion-exchanged water. The solid content was allowed to dry at 120° C. to obtain silver nitrate supporting silica. The silver nitrate supporting silica was mixed with 200 mL of 1M hydrochloric acid, which was then stirred for four hours. The solid content was filtered and washed with ion-exchanged water. The solid content was allowed to dry at 120° C. to obtain silver chloride supporting silica. The content of the silver chloride in the silver chloride supporting silica was 20%.

A polystyrene resin (EH504H manufactured by Kuraray Co., Ltd.) was dissolved in toluene, and silver (AG-4-8F manufactured by DOWA Electronics Materials Co., Ltd., particle size: 1.9 μm) and the silver chloride carrying silica having the silver chloride content of 20%, which had been prepared as above, were dispersed therein to prepare a dispersion.

The entire surface of the polymerizable electrode layer was covered with the above-prepared dispersion by hand coating using a film applicator, and then allowed to dry at 120° C. to form a non-polarizable electrode layer.

The substrate on which the polarizable electrode layer and the non-polarizable electrode layer were formed was cut into a piece of 15×30 mm; a conductive gel layer (CR-H manufactured by Sekisui Kasei Co., Ltd.) having a size of 15×15 mm was attached to a surface of the non-polarizable electrode layer so as to have one end edge of the conductive gel layer overlapping a shorter end edge of the substrate; and a lead wire was placed to be sandwiched between these layers for fixing, to obtain a biological electrode.

### Comparative Example 1

A biological electrode was manufactured in the same manner as in Example 1, except that silica was changed to carbon particles (D50: 3.3 μm). The silver chloride content in the silver chloride supporting carbon was 20%.

### Comparative Example 2

A biological electrode was manufactured in the same manner as in Example 1, except that silver chloride was used as was, without being supported by silica.

### Evaluation Method

The biological electrode was sealed in an aluminum pack, which was stored in a constant temperature bath set to 57° C. Storage at 57° C. for 10 weeks is considered to correspond to storage at room temperature for 2 years. After a specific period of time elapsed, the biological electrode was taken out of the aluminum pack, and was subjected to measurements of impedance (ACZ) and polarization after defibrillation load (SDR/DCO) in accordance with the method of ANSI/AAMI EC12:2000 to thereby evaluate the electrical properties of the biological electrode. With the ACZ being 750Ω or less and the SDR/DCO being 20 mV or less as references, a judgment was made as to whether the biological electrode was excellent in electrical performance.

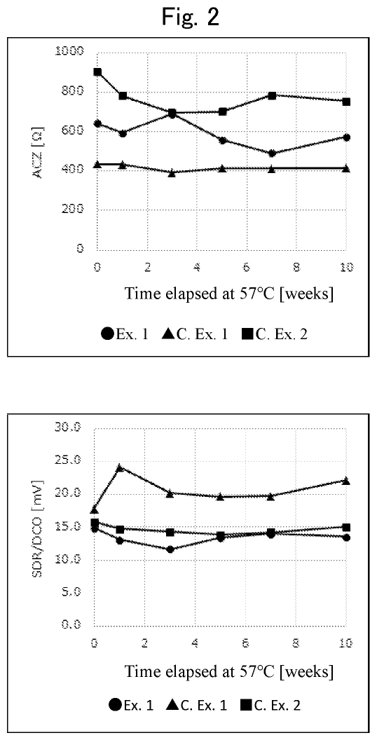

As shown in Table 1 and FIG. 2, Example 1 was found to be excellent in electrical performance with both the ACZ and the SDR/DCO falling below the above references. In contrast, Comparative Example 1 had some cases where the SDR/DCO exceeded the above reference and Comparative Example 2 had some cases where the ACZ exceeded the above reference, which were found to have poorer electrical performance than Example 1.

TABLE 1

| | | Ex. 1 | C. Ex. 1 | C. Ex. 2 |
|---|---|---|---|---|
| Composition | Resin | 100 mass | 100 mass | 100 mass |
| | | parts | parts | parts |
| | Silver | 180 mass | 180 mass | 180 mass |
| | | parts | parts | parts |
| | Silver chloride | 10 mass | 10 mass | 10 mass |
| | | parts | parts | parts |
| | Silver:Silver | 95:5 | 95:5 | 95:5 |
| | chloride | | | |
| | Silica | 40 mass | — | — |
| | | parts | | |
| | Carbon particles | — | 40 mass | — |
| | | | parts | |
| Layer | Non-polarizable | 3 | 3 | 3 |
| thickness | electrode layer | | | |
| (μm) | Polarizable | 5 | 5 | 5 |
| | electrode layer | | | |
| ACZ (Ω) | 0 week | 642 | 436 | 905 |
| | 1 week | 595 | 433 | 782 |
| | 3 weeks | 690 | 394 | 697 |
| | 5 weeks | 560 | 415 | 703 |
| | 7 weeks | 492 | 414 | 784 |
| | 10 weeks | 574 | 416 | 755 |
| SDR/DCO | 0 week | 15.0 | 17.8 | 15.8 |
| (mV) | 1 week | 13.2 | 24.2 | 14.8 |
| | 3 weeks | 11.7 | 20.2 | 14.4 |
| | 5 weeks | 13.4 | 19.7 | 13.9 |
| | 7 weeks | 14.0 | 19.8 | 14.3 |
| | 10 weeks | 13.6 | 22.2 | 15.1 |

Next, the content of silver was evaluated based on the mixing ratio of Example 1. The results are shown in Table 2. Comparative Example 3, in which the content of silver was 140 mass parts, had some cases where the ACZ exceeded the above reference, and was found to have poorer electrical performance than Examples 1 to 4.

TABLE 2

| | | Ex. 1 | Ex. 2 | Ex. 3 | Ex. 4 | C. Ex. 3 |
|---|---|---|---|---|---|---|
| Composition | Resin | 100 mass | 100 mass | 100 mass | 100 mass | 100 mass |
| | | parts | parts | parts | parts | parts |
| | Silver | 180 mass | 250 mass | 150 mass | 300 mass | 140 mass |
| | | parts | parts | parts | parts | parts |
| | Silver chloride | 10 mass | 10 mass | 6 mass | 10 mass | 8 mass |
| | | parts | parts | parts | parts | parts |
| | Silver:Silver | 95:5 | 96:4 | 96:4 | 97:3 | 95:5 |
| | chloride | | | | | |
| | Silica | 40 mass | 40 mass | 24 mass | 40 mass | 32 mass |
| | | parts | parts | parts | parts | parts |
| Layer | Non-polarizable | 3 | 3 | 3 | 3 | 3 |
| thickness | electrode layer | | | | | |
| (μm) | Polarizable | 5 | 5 | 10 | 5 | 5 |
| | electrode layer | | | | | |
| ACZ (Ω) | 0 week | 642 | 709 | 678 | 495 | 968 |
| | 1 week | 595 | 703 | 376 | 459 | 766 |
| | 3 weeks | 690 | 663 | 382 | 473 | 648 |
| | 5 weeks | 560 | 473 | 349 | 427 | 783 |
| | 7 weeks | 492 | 406 | 469 | 377 | 670 |
| | 10 weeks | 574 | 435 | 367 | 374 | 853 |
| SDR/DCO | 0 week | 15.0 | 13.8 | 678 | 13.9 | 16.3 |
| (mV) | 1 week | 13.2 | 14.3 | 376 | 15.0 | 16.6 |
| | 3 weeks | 11.7 | 12.7 | 382 | 14.4 | 14.4 |
| | 5 weeks | 13.4 | 13.7 | 349 | 14.8 | 13.7 |
| | 7 weeks | 14.0 | 12.5 | 469 | 13.5 | 15.9 |
| | 10 weeks | 13.6 | 13.8 | 367 | 13.7 | 15.8 |

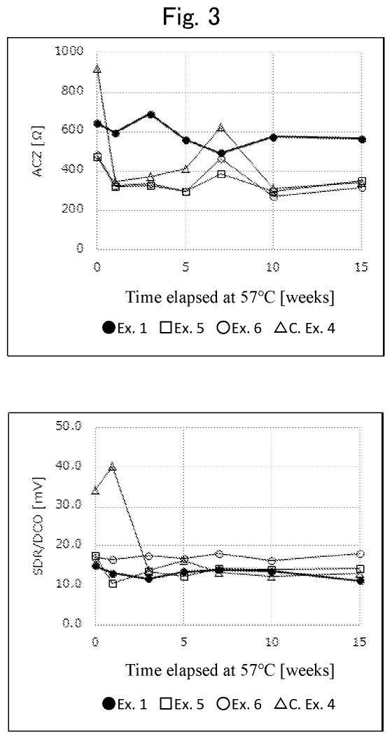

Next, the ratio between the content of silver and the content of silver chloride was evaluated. The results are shown in Table 3 and FIG. 3. Comparative Example 4, in which silver:silver chloride=98:2, was found to have poor electrical performance with both the ACZ and the SDR/DCO exceeding the references.

TABLE 3

| | | Ex. 1 | Ex. 5 | Ex. 6 | C. Ex. 4 |
|---|---|---|---|---|---|
| Compo- | Resin | 100 mass | 100 mass | 100 mass | 100 mass |
| sition | | parts | parts | parts | parts |
| | Silver | 180 mass | 180 mass | 180 mass | 180 mass |
| | | parts | parts | parts | parts |
| | Silver | 10 mass | 9 mass | 7.2 mass | 3.6 mass |
| | chloride | parts | parts | parts | parts |
| | Silver:Silver | 95:5 | 95:5 | 96:4 | 98:2 |
| | chloride | | | | |
| | Silica | 40 mass | 36 mass | 28.8 mass | 14.4 mass |
| | | parts | parts | parts | parts |
| Layer | Non- | 3 | 3 | 3 | 3 |
| thickness | polarizable | | | | |
| (μm) | electrode | | | | |
| | layer | | | | |
| | Polarizable | 5 | 10 | 10 | 10 |
| | electrode | | | | |
| | layer | | | | |
| ACZ (Ω) | 0 week | 642 | 383 | 474 | 918 |
| | 1 week | 595 | 290 | 321 | 347 |
| | 3 weeks | 690 | 315 | 325 | 374 |
| | 5 weeks | 560 | 347 | 296 | 414 |
| | 7 weeks | 492 | 338 | 385 | 622 |
| | 10 weeks | 574 | 358 | 295 | 311 |
| SDR/DCO | 0 week | 15.0 | 16.6 | 17.5 | 34.3 |
| (mV) | 1 week | 13.2 | 19.5 | 10.4 | 40.1 |
| | 3 weeks | 11.7 | 12.6 | 13.5 | 13.9 |
| | 5 weeks | 13.4 | 13.9 | 12.3 | 16.2 |
| | 7 weeks | 14.0 | 13.0 | 14.4 | 13.3 |
| | 10 weeks | 13.6 | 14.3 | 14.0 | 12.4 |

Next, the thicknesses of the non-polarizable electrode layer and the polarizable electrode layer were evaluated based on the mixing ratio of Example 1. The results are shown in Table 4. Comparative Example 5, in which the thickness of the non-polarizable electrode layer was set to 2 had the SDR/DCO exceeding the reference, and Comparative Example 6, in which the thickness of the non-polarizable electrode layer was set to 8 had the ACZ greatly exceeding the reference, both of which were found to have poor electrical performance. It is conceivable from these results that the thickness of the non-polarizable electrode layer be preferably more than 2 μm and less than 8 μm.

Also, radiography of the biological electrode of each of Example 1 and Example 5 revealed that the electrode was hardly visible in an x-ray image.

TABLE 4

| | | Ex. 1 | Ex. 5 | C. Ex. 5 | C. Ex. 6 |
|---|---|---|---|---|---|
| Compo- | Resin | 100 mass | 100 mass | 100 mass | 100 mass |
| sition | | parts | parts | parts | parts |
| | Silver | 180 mass | 180 mass | 180 mass | 180 mass |
| | | parts | parts | parts | parts |
| | Silver | 10 mass | 9 mass | 10 mass | 10 mass |
| | chloride | parts | parts | parts | parts |
| | Silver:Silver | 95:5 | 95:5 | 95:5 | 95:5 |
| | chloride | | | | |
| | Silica | 40 mass | 36 mass | 40 mass | 40 mass |
| | | parts | parts | parts | parts |
| Layer | Non- | 3 | 3 | 2 | 8 |
| thickness | polarizable | | | | |
| (μm) | electrode | | | | |
| | layer | | | | |
| | Polarizable | 5 | 10 | 5 | 5 |
| | electrode | | | | |
| | layer | | | | |
| ACZ (Ω) | 0 week | 642 | 383 | 595 | 2100 |
| | 1 week | 595 | 290 | 647 | 874 |
| | 3 weeks | 690 | 315 | 574 | 717 |
| | 5 weeks | 560 | 347 | 604 | — |
| | 7 weeks | 492 | 338 | 590 | — |
| | 10 weeks | 574 | 358 | 609 | — |
| SDR/DCO | 0 week | 15.0 | 16.6 | 24.6 | 18.3 |
| (mV) | 1 week | 13.2 | 19.5 | 11.6 | 17.3 |
| | 3 weeks | 11.7 | 12.6 | 12.5 | 16.5 |
| | 5 weeks | 13.4 | 13.9 | 11.5 | — |
| | 7 weeks | 14.0 | 13.0 | 12.3 | — |
| | 10 weeks | 13.6 | 14.3 | 16.1 | — |

From the above results, the thickness of the polarizable electrode layer is found to greatly contribute to the ACZ value, and the ACZ value may exceed the reference particularly when the thickness falls below 5 μm. Thus, it is conceivable that the thickness of the polarizable electrode layer be 5 μm or more.

### REFERENCE SIGNS LIST

1: Biological electrode

10: Substrate

20: Electrode layer

22: Polarizable electrode layer

24: Non-polarizable electrode layer

26: Conductive gel layer

30: Lead wire

1. A biological electrode comprising:
a polarizable electrode layer; and
a non-polarizable electrode layer laminated on the polarizable electrode layer, wherein
the non-polarizable electrode layer comprises a resin, silver, and silver chloride supported by silica,
a content of the silver is 150 to 300 mass parts based on 100 mass parts of the resin,
a ratio between the silver and the silver chloride is 97:3 to 95:5, and
the non-polarizable electrode layer has a thickness of 3 to 5 μm.

2. The biological electrode according to claim 1, wherein a content of the silica is 20 to mass parts based on 100 mass parts of the resin.

3. The biological electrode according to claim 1, wherein the polarizable electrode layer has a thickness of 5 to 10 μm.
